(19) United States
(12) Patent Application Publication
(10) Pub. No.: US 2017/0136010 A1
(43) Pub. Date: May 18, 2017

(54) ORAL SOLID PREPARATION COMPRISING ARIPIPRAZOLE AND METHOD FOR PRODUCING ORAL SOLID PREPARATION COMPRISING ARIPIPRAZOLE

(71) Applicant: OTSUKA PHARMACEUTICAL CO., LTD, Tokyo (JP)

(72) Inventors: Haruka YOSHIDA, Osaka (JP); Toshiaki TANIGUCHI, Osaka (JP); Tadashi MUKAI, Osaka (JP)

(73) Assignee: OTSUKA PHARMACEUTICAL CO., LTD, Tokyo (JP)

(21) Appl. No.: 15/418,595

(22) Filed: Jan. 27, 2017

Related U.S. Application Data
(63) Continuation of application No. 14/265,883, filed on Apr. 30, 2014.

(30) Foreign Application Priority Data
Sep. 30, 2013 (JP) 2013-204546

(51) Int. Cl. A61K 31/496; A61K 9/16; A61K 9/00; A61K 9/20

(52) U.S. Cl. CPC A61K 31/496; A61K 9/2095; A61K 9/1688; A61K 9/0053

(57) ABSTRACT

[Object] An object of the present invention is to provide an oral solid preparation that can be produced in a simpler manner than conventional methods, that exhibits high bioavailability and high dissolubility even in persons having low stomach acid, and that can also ensure dissolubility after being allowed to stand for a certain period of time. Another object is to provide a simple method for producing the oral solid preparation.

[Means for Achieving the Object] The present invention relates to an oral solid preparation comprising, as an active ingredient, a finely milled powder obtained by milling a highly hygroscopic aripiprazole anhydrous crystal, and a pharmaceutically acceptable carrier, the finely milled powder having a mean particle size of 10 μm or less; and a method for producing an oral solid preparation comprising the steps of (1) milling a highly hygroscopic aripiprazole anhydrous crystal into a finely milled powder having a mean particle size of 10 μm or less, and (2) mixing the obtained finely milled powder with a pharmaceutically acceptable carrier.

### TECHNICAL FIELD

The present invention relates to an oral solid preparation comprising aripiprazole as an active ingredient, and a method for producing an oral solid preparation comprising aripiprazole as an active ingredient.

### BACKGROUND ART

Aripiprazole, i.e., 7-{4-[4-(2,3-dichlorophenyl)-1-piperazinyl]butoxy}-3,4-dihydrocarbostyril or 7-{4-[4-(2,3-dichlorophenyl)-1-piperazinyl]butoxy}-3,4-dihydro-2(1H)-quinolinone, is known as an atypical antipsychotic that is useful for the treatment of schizophrenia, and aripiprazole anhydrous crystals are also known (Patent Document 1: JP1990-191256A and Non-patent Document 1: Proceedings of the Fourth Japan-Korea Joint Symposium on Separation Technology).

The aripiprazole anhydrous crystals have high hygroscopicity, and are therefore problematic in various ways (paragraph [0006] of Patent Document 2). One such problem is that, due to their high hygroscopicity, aripiprazole anhydrous crystals are prone to hydration. Furthermore, compared to anhydrous crystals, hydrated crystals are low in bioavailability and dissolubility. In particular, hydrated crystals exhibit low bioavailability and dissolubility in people with low stomach acid. A high proportion of Asian people have low stomach acid. In Japanese, who are of Asian ethnicity, 25% of people 60 years of age or older are said to have low stomach acid.

There are also other problems caused by high hygroscopicity. For example, Patent Document 2 describes, as problems, the variation in the amount of aripiprazole hydrate versus anhydrous aripiprazole from batch to batch; increased packaging cost due to concerns regarding moisture absorption during storage and handling; poor storage stability resulting from reduction of packaging cost; etc. (paragraph [0006] of Patent Document 2).

The aripiprazole anhydrous crystals disclosed in Patent Document 1 and Non-patent Document (NPL) 1 have problematically high hygroscopicity; however, such aripiprazole anhydrous crystals exhibit high bioavailability in persons having low stomach acid, and also have high dissolubility. Therefore, various research and analysis has been conducted on low hygroscopic aripiprazole anhydrous crystals.

As a result of such research and analysis, a novel low hygroscopic aripiprazole anhydrous crystal B that is resistant to hydration was successfully obtained from a novel aripiprazole hydrate A (Patent Document 2).

The low hygroscopic aripiprazole anhydrous crystal F can solve the above-mentioned various problems.

Patent Document 2 discloses that the novel aripiprazole anhydrous crystal B is produced by a method comprising milling a known aripiprazole hydrate (preferably monohydrate) to form a novel aripiprazole hydrate A, and heating the hydrate A to form a novel aripiprazole anhydrous crystal B.

### PRIOR ART DOCUMENTS

### Patent Documents

[Patent Document 1] JP1990-191256A (Japanese Patent No. 2608788)
[Patent Document 2] Japanese Patent No. 3760264

### Non-Patent Document

[Non-patent Document 1] Satoshi Aoki et al., “Study on Crystal Transformation of Aripiprazole,” Proceedings of the Fourth Japan-Korea Joint Symposium on Separation Technique (held on Oct. 6 to 8, 1996), pages 937 to 940 (issued in Japan)

### SUMMARY OF INVENTION

### Problem to be Solved by the Invention

However, the production process disclosed in the above Patent Document 2 comprises many production steps. If an oral solid preparation that exhibits high bioavailability and dissolubility in persons having low stomach acid can be produced in a simple manner, time and cost can be reduced.

Accordingly, an object of the present invention is to provide an oral solid preparation that can be produced in a manner simpler than conventional methods, that exhibits high bioavailability and high dissolubility even in persons having low stomach acid, and that can also ensure dissolubility after being allowed to stand for a certain period of time.

Another object of the present invention is to provide a simple method for producing an oral solid preparation that has high bioavailability and high dissolubility, and that can also ensure dissolubility after being allowed to stand for a certain period of time.

### Means for Solving the Problem

As a result of extensive research, the present inventors achieved the above objects by attempting the use of a highly hygroscopic aripiprazole anhydrous crystal which had been considered inapplicable to medicine by persons skilled in the art, i.e., an aripiprazole anhydrous crystal that is highly hygroscopic and therefore exhibits reduced dissolubility after storage for a certain period of time.

More specifically, the present invention uses a finely milled aripiprazole crystal obtained by simply milling a highly hygroscopic aripiprazole anhydrous crystal to a mean particle size in a specific range, and can thereby provide an oral solid preparation that can ensure dissolubility after being allowed to stand for a certain period of time, and that has high bioavailability and dissolubility equivalent to those of an oral solid preparation comprising aripiprazole anhydrous crystal B.

Further, the present invention can provide a simple production method in which a finely milled crystal obtained by simply milling a highly hygroscopic aripiprazole anhydrous crystal is directly used for the production of an oral solid preparation. For example, unlike the production process disclosed in Patent Document 2, the step of heating aripiprazole hydrate A for drying can be omitted.

The present invention provides oral solid preparations according to items 1 to 6, and methods for producing the oral solid preparations.

Item 1. An oral solid preparation comprising, as an active ingredient, a finely milled crystal obtained by milling a highly hygroscopic aripiprazole anhydrous crystal, and a pharmaceutically acceptable carrier, the finely milled crystal having a mean particle size of 10 μm or less.
Item 2. The oral solid preparation according to item 1, wherein the finely milled crystal has a mean particle size of 8 μm or less.
Item 3. The oral solid preparation according to item 1, wherein the finely milled crystal has a mean particle size of 6 μm or less.
Item 4. The oral solid preparation according to any one of items 1 to 3, wherein the finely milled crystal has a mean particle size of 1 μm or more.
Item 5. A method for producing an oral solid preparation comprising the steps of:
(1) milling a highly hygroscopic aripiprazole anhydrous crystal into a finely milled crystal having a mean particle size of 10 μm or less; and
(2) mixing the obtained finely milled crystal with a pharmaceutically acceptable carrier.
Item 6. The method for producing an oral solid preparation according to item 5, wherein the oral solid preparation is a tablet, and the method further comprises the step of (3) compressing the mixture obtained in step (2) into tablets.

### Effect of the Invention

The present invention can provide an oral solid preparation that can be produced in a simpler manner than conventional methods, that exhibits high bioavailability and high dissolubility even in people having low stomach acid, and that can also ensure dissolubility after being allowed to stand for a certain period of time.

The present invention can further provide a simple method for producing an oral solid preparation that can ensure dissolubility after being allowed to stand for a certain period of time and that therefore has high bioavailability and high dissolubility.

According to the present invention, the mean particle size of the particles of the highly hygroscopic aripiprazole anhydrous crystal as an active ingredient is set to a specific low range. This enables the production an oral solid preparation which is not affected by the external environment (e.g., external humidity) and whose dissolubility after being allowed to stand for a certain period of time is not impaired.

Further, the oral solid preparation of the present invention does not require the use of an aluminum pouch when packing the pharmaceutical in a blister pack (a PTP film), or a desiccant when sealing the pharmaceutical in a bottle.

Additionally, when using the method for producing the oral solid preparation of the present invention, the oral solid preparation obtained by the method is not affected by hygroscopicity of highly hygroscopic aripiprazole anhydrous crystal as an active ingredient; the oral solid preparation exhibits high dissolubility immediately after production, and dissolubility of the preparation after being allowed to stand for a certain period of time is also not impaired. Therefore, for example, when the preparation is formed into tablets, usable methods are not only a wet granule compression method or like production methods that comprise the steps of first preparing granules having a controlled water content, and then drying to reduce the water content of the granules; tablets can also be easily produced by other compression methods, such as a direct compression method or a dry granule compression method.

### MODE FOR CARRYING OUT THE INVENTION

The expression “comprising” used herein includes the concepts of “comprising,” “essentially consisting of,” and “consisting of.”

### 1. Oral Solid Preparation

The oral solid preparation of the present invention comprises, as an active ingredient, a highly hygroscopic aripiprazole anhydrous crystal having a mean particle size of 10 μm or less, and a pharmaceutically acceptable carrier.

The highly hygroscopic aripiprazole anhydrous crystals that can be used in the present invention are not particularly limited, and any crystals may be used. Examples of highly hygroscopic aripiprazole anhydrous crystals include the anhydrous crystals disclosed in Reference Examples 1 and 2 of Patent Document 2 (Japanese Patent No. 3760264), the aripiprazole anhydrous crystals disclosed in Patent Document 1 (JP1990-191256A), and aripiprazole Type I disclosed in Non-patent Document 1.

Further, the method for producing a highly hygroscopic aripiprazole anhydrous crystal is not particularly limited, and known production methods can be used, Examples of usable methods include a process disclosed in Example 1 of Patent Document 1 (JP1990-191256A), which comprises reacting 7-(4-bromobutoxy)-3,4-dihydrocarbostyril with 1-(2,3-dichlorophenyl)piperazine, and production processes disclosed in Patent Document 2 and Non-patent Document 1.

The highly hygroscopic aripiprazole anhydrous crystals are, for example, aripiprazole anhydrous crystals that have a hygroscopicity such that the water content after being allowed to stand in a desiccator at a temperature of 60° C. and a humidity of 100% for 24 hours is more than 0.40%. The upper limit of the water content is preferably 4.5% or less, and more preferably 3.9% or less.

Upon exposure to moisture, highly hygroscopic conventional aripiprazole anhydrous crystals gain moisture, and convert into aripiprazole hydrates. Therefore, these crystals tended to be incapable of ensuring dissolubility with the lapse of time after production of the preparation. However, mean particle size reduction can provide a remarkable effect, i.e., ensuring a high dissolution rate of tablets even after being allowed to stand for a certain period of time in a dissolution test, in spite of the conversion of aripiprazole anhydrous into aripiprazole hydrate due to moisture.

The mean particle size of highly hygroscopic aripiprazole anhydrous crystals is about 10 μm or less, preferably 8 μm or less, and more preferably about 6 μm or less. Setting the mean particle size of highly hygroscopic aripiprazole anhydrous crystals to about 10 μm or less can provide an oral solid preparation that has high dissolubility immediately after production and also ensure dissolubility that is not impaired even after being allowed to stand for a certain period of time.

Setting the mean particle size to the above-mentioned specific range can achieve a dissolution rate of about 60% or more at pH 5 after 60 minutes (the dissolution rate that can maintain the maximum blood concentration (Cₘₐₓ)) in dissolution tests performed immediately after the production of the oral solid preparation, and after lapse of time.

The mean particle size of highly hygroscopic aripiprazole anhydrous crystals is not particularly limited. However, the mean particle size is preferably about 1 μm or more, more preferably about 2 μm or more, and most preferably about 2.5 μm or more, from the viewpoint of preventing an increase of the mean particle size due to reaggregation, reducing costs while not requiring excessive time to mill aripiprazole crystals, and facilitating handling. In particular, setting the mean particle size of aripiprazole anhydrous crystals to 2 μm or more, more preferably about 2.5 μm or more is preferable because it prevents dissolubility variation of aripiprazole anhydrous crystals as an active ingredient, and can stably provide an oral solid preparation with excellent dissolubility.

More specifically, the aripiprazole anhydrous crystals preferably have a mean particle size of about 1 to 10 μm (particularly about 2 to 10 μm, and more particularly about 2.5 to 10 μm), more preferably about 1 to 8 μm (particularly about 2 to 8 μm, and more particularly about 2.5 to 8 μm), and even more preferably about 1 to 6 μm (particularly about 2 to 6 μm, and more particularly about 2.5 to 6 μm).

The term “mean particle size” refers to volume mean diameter as calculated from the particle size distribution measured by a laser scattering particle size distribution analyzer analyzer.

The particle size ratio of the specific particle size of particles can be calculated from particle size distribution measured by a laser scattering particle size distribution analyzer.

The mean particle size measurement by the above method can also be performed using a light microscope. The measurement can be performed in accordance with the method described in the Japanese Pharmacopoeia (for example, B. General Tests, 3.04 Particle Size Determination, 1. First Optical Microscopy, described in the Handbook of the Japanese Pharmacopoeia, 16th Edition, Health, Labor and Welfare Ministry, Notification 65 (Mar. 24, 2011)). The mean particle size measured according to the method described in the Japanese Pharmacopoeia may be, for example, in the range of 3 to 15 μm.

The oral solid preparation preferably comprises the aripiprazole crystal in an amount of about 0.5 to 80 wt. %, and more preferably about 1 to 70 wt. %.

Examples of pharmaceutically acceptable carriers in the oral solid preparation of the present invention include diluents and excipients generally used in pharmaceuticals, such as fillers, extenders, binders, humectants, disintegrators, surfactants, and lubricants.

The pharmaceutical composition of the present invention may be in the form of a general pharmaceutical preparation. Examples of such forms include tablets, flash-melt tablets, pills, powders, granules, capsules, and the like.

When the preparation is formed into tablets, a wide variety of carriers conventionally known in this field can be used. Examples of such carriers include excipients such as lactose, sucrose, sodium chloride, glucose, urea, starch, xylitol, mannitol, erythritol, sorbitol, calcium carbonate, kaolin, crystalline cellulose, and silicic acid; binders such as water, ethanol, propanol, simple syrup, glucose solution, starch solution, gelatin solution, carboxymethyl cellulose, shellac, methylcellulose, potassium phosphate, and polyvinylpyrrolidones; disintegrators such as dry starch, sodium alginate, agar powder, laminaran powder, sodium hydrogen carbonate, calcium carbonate, polyoxyethylene sorbitan fatty acid esters, sodium lauryl sulfate, stearic acid monoglyceride, starch, and lactose; disintegration inhibitors such as sucrose, stearin, cacao butter, and hydrogenated oils; absorption enhancers such as quaternary ammonium base and sodium lauryl sulfate; humectants such as glycerol and starch; adsorbents such as starch, lactose, kaolin, bentonite, and colloidal silica; and lubricants such as purified talc, stearate, boric acid powder, and polyethylene glycol. Tablets can also be formed as tablets with ordinary coatings, if necessary. Examples of such tables include sugar-coated tablets, gelatin-coated tablets, enteric-coated tablets, film-coated tablets, double-layer tablets, and multilayer tablets.

When the preparation is formed into pills, a wide variety of carriers conventionally known in this field can be used. Examples of usable carriers include excipients such as glucose, lactose, starch, cacao butter, hydrogenated vegetable oil, kaolin, and talc; binders such as powdered gum arabic, powdered tragacanth, gelatin, and ethanol; disintegrators such as laminaran and agar; and the like.

Capsules are prepared according to ordinary methods by mixing a highly hydroscopic aripiprazole anhydrous crystal with one or more of various carriers as exemplified above, and packing the mixture in hard gelatin capsules, soft capsules, hydroxypropylmethyl cellulose capsules (HPMC capsules), or the like.

If necessary, colorants, preservatives, perfumes, flavorings, sweeteners, and the like, as well as other drugs may be incorporated into the pharmaceutical composition.

When the solid oral preparation is formed into granules, a liquid (typically, water or an aqueous solution containing a binder) is added to a mixed powder of granulating ingredients comprising a highly hygroscopic aripiprazole anhydrous crystal and a carrier. As the carrier, various carriers known in this field can be used. Examples of such carriers include excipients, disintegrators, disintegration inhibitors, humectants, absorption enhancers, adsorbents, lubricants, colorants, and the like. Specific examples thereof include those mentioned above.

The oral solid preparation of the present invention may have any dissolution rate that is about 60% or more at pH 5 after 60 minutes as measured by a dissolution test method. When the dissolution rate at pH 5 after 60 minutes is about 60% or more, the active ingredient in the oral solid preparation administered can be sufficiently absorbed into the body, and the maximum blood concentration (Cₘₐₓ) can be maintained. Therefore, a dissolution rate of about 60% or more is preferable.

The dissolution test method is in accordance with the dissolution test method (paddle method) in the Japanese Pharmacopoeia, Sixteenth Edition, and is a method as described below.

One tablet of an oral solid preparation is added to 500 ml of acetate buffer (pH 5.0), which is used as a test liquid, thereby obtaining a test solution. Using the test solution, a dissolution test is performed at a paddle speed of 75 rpm in accordance with the method 2 (paddle method) for dissolution test. As the acetate buffer, a solution prepared by adding water to 1.97 g of acetic acid and 9.15 g of sodium acetate trihydrate to make 1000 ml of solution is used.

A standard solution is prepared in the following manner. 0.05 g of a standard sample of aripiprazole is weighed accurately, and ethanol (95%) is added to make exactly 50 ml of solution. Then, 6 ml of this solution is weighed accurately, and the test liquid is added to make exactly 1000 ml of solution.

The test solutions and the standard solution are subjected to filtration using a filter having a pore size of 10 or 20 μm. Each of the filtrates is introduced to a spectrophotometer equipped with a flow cell (cell length: 10 mm), and tested by UV visible absorbance spectroscopy. The differences between the absorbance of each solution at a wavelength of 249 nm and the absorbance thereof at a wavelength of 325 nm, which are At10, At20, At30, At45, At60, and As, are determined. At10, At20, At30, At45, and At60 indicate differences between the absorbances of each test solution at 10, 20, 30, 45, and 60 minutes after the beginning of the dissolution test, respectively. As indicates a difference between the absorbances of the standard solution.

The dissolution rate (%) relative to the indicated amount of aripiprazole is determined by the following formula:

Dissolution Rate (%) relative to the indicated amount of aripiprazole=Amount of the aripiprazole standard product (mg)×At×As×2

In the Formula, As indicates a difference between the absorbances of the standard solution, and At indicates At10, At20, At30, At45, or At60.

More specifically, the measurement of the dissolution rate by the above dissolution test method can be performed by the method shown in Test Example 1 of the Examples below.

The method for administering the oral solid preparation of the present invention is not particularly limited. The preparation can be orally administered by a method suitable for the patient's age, sex, and other conditions (e.g., severity of disease).

The dosage of the oral solid preparation of the present invention can be suitably selected according to the dose regimen, the patient's age, sex, and other conditions (e.g., severity of disease), etc. Generally, the preparation is administered in an amount such that the amount of aripiprazole, which is an active ingredient, is about 0.1 to 10 mg per kg of body weight per day. The amount of aripiprazole per tablet of the oral solid preparation is in the range of about 1 to 100 mg, and preferably about 1 to 30 mg.

The oral solid preparation of the present invention is effective in the prevention and treatment of schizophrenia, including intractable (drug-resistant, chronic) schizophrenia with cognitive impairment and intractable (drug-resistant, chronic) schizophrenia without cognitive impairment; anxiety, including mild anxiety; mania, including acute mania such as bipolar disorder acute mania; bipolar disorder; depression, including bipolar disorder depression; autism, Down's syndrome, attention deficit hyperactivity disorder (ADHD)), Alzheimer's disease, Parkinson's disease, and like neurodegenerative diseases; panic, obsessive compulsive disorder (OCD), sleep disorder, sexual dysfunction, alcohol and drug dependency, vomiting, motion sickness, obesity, migraine, cognitive impairment, and like central nervous system diseases; and major depression, behavioral and psychological symptoms of dementia (BPSD), Tourette's syndrome, bulimia nervosa, and chronic pain/fibromyalgia/chronic fatigue syndrome.

### 2. Method for Producing the Oral Solid Preparation

The method for producing an oral solid preparation of the present invention comprises the steps of (1) milling a highly hygroscopic aripiprazole anhydrous crystal to obtain a finely milled crystal having a mean particle size of 10 μm or less, and (2) mixing the obtained finely milled crystal with a pharmaceutically acceptable carrier.

The finely milled crystal obtained by milling in step (1) can be directed subjected to the subsequent step (2). For example, without drying after milling, the finely milled crystal can be used to produce the oral solid preparation.

Examples of the highly hygroscopic aripiprazole anhydrous crystal used in step (1) may be the same as the anhydrous crystals mentioned above in “1. Oral solid preparation.”

The method for milling the highly hygroscopic aripiprazole crystal in step (1) is not particularly limited, and may be wet milling or dry milling. Various milling methods and mills can be used. The milling step can be performed by suitably selecting or suitably combining the conditions, devices, and methods that can produce a highly hygroscopic aripiprazole anhydrous crystal having a mean particle size within the specific range of the present invention. From the viewpoint of ease of operation, dry milling is preferable.

When the milling in step (1) is performed by a dry milling method, examples of usable mills include jet mills, ball mills (e.g., Dyno mills), other low-energy mills (e.g., roller mills), and high-energy mills. Examples of high-energy mills include Netzsch mills, DC mills, planetary mills, and the like.

In particular, when a jet mill is used as a mill, the mean particle size of the milled highly hygroscopic aripiprazole anhydrous crystal can be set by two types of air pressure (“forcing pressure” for feeding the powder to the milling chamber, and “milling pressure” of air directly sent to the milling chamber) and the “feed rate of the powder.” Generally, as the air pressure is set high and the feed rate of the powder is set low, milling strength increases and the particle size tends to be small.

When the milling in step (1) is performed by wet milling, usable mills include, for example, high-pressure homogenizers and bead mills.

Step (2) is a step of mixing the highly hygroscopic aripiprazole anhydrous crystal milled in step (1), and a pharmaceutically acceptable carrier.

Examples of pharmaceutically acceptable carriers used in step (2) may be the same as those mentioned above in “1. Oral solid preparation.”

The mixing method in step (2) is not particularly limited as long as the milled highly hygroscopic aripiprazole anhydrous crystal can be mixed with pharmaceutically acceptable carriers, such as excipients, binders, disintegrators, disintegration inhibitors, absorption enhancers, humectants, and adsorbents.

The powder of the highly hygroscopic aripiprazole anhydrous crystal and the powder of a pharmaceutically acceptable carrier may be mixed by dry granulation, or may be mixed by wet granulation and formed into granules.

For wet granulation, various methods such as a fluidized bed granulation method, kneading granulation method, extrusion granulation method, and rotating granulation method can be used.

For example, when using a fluidized bed granulation method, granulating ingredients comprising various carriers are mixed by supplied air; while the granulating ingredients are continuously allowed to flow, a liquid is sprayed thereover to form granules. When using a kneading granulation method, granulating ingredients comprising various carriers are mixed by stirring; while the granulating ingredients are continuously stirred, a liquid is added thereto to form granules.

After the granulation, the obtained granules are, if necessary, passed through a screen to make the granules a desired size. The granules thus obtained are subjected to a drying process.

A wide variety of known methods can be used as the drying method. Examples of usable driers include fluidized bed dryers, fan dryers, vacuum dryers, and the like. For example, when using a fluidized bed dryer, the drying procedure is conducted with an air flow of 0.5 m³/min to 50 m³/min, and a supplied air temperature of 70 to 100° C. for about 10 minutes to 1 hour.

The content of the highly hygroscopic aripiprazole anhydrous crystal in the mixture obtained in step (2) is preferably set to the same range as mentioned above in “1, Oral solid preparation.”

When the oral solid preparation is formed into tablets by the method for producing the oral solid preparation of the present invention, the method further comprises a step of compressing the mixture obtained in step (2) into tablets.

Further adding a lubricant to the mixture obtained in step (2) is preferable in view of inhibiting the adhesion of the raw powder to a tableting machine during compression tableting in the subsequent step (3).

Various compression methods can be used in the compression tableting of step (3). Examples of usable methods include a wet granule compression method, direct powder compression method, dry granule compression method, and the like. Obtaining tablets by a direct powder compression method (direct compression method) is preferable in view of simple production of tablets that does not involve a complicated step of first preparing granules as in a wet granule compression method or dry granule compression method. Generally, when an oral solid preparation containing aripiprazole as an active ingredient is obtained by a direct compression method, low dissolubility tends to result. In contrast, in the present invention, because aripiprazole anhydrous crystal as an active ingredient has a mean particle size within a specific range, a desired oral solid preparation comprising aripiprazole anhydrous crystal, whose dissolubility is not reduced even when produced by a direct compression method, can be obtained.

When the oral solid preparation obtained by the production method of the present invention is formed into tablets, the tablets may be in the form of bilayer tablets, multilayer tablets, etc.

As long as high dissolubility, which is the effect of the present invention, is not impaired, a step of further coating may be performed, if necessary. For example, sugar-coated tablets, gelatin-coated tablets, enteric-coated tablets, and film-coated tablets can be produced.

The oral solid preparation obtained by the production method of the present invention exhibits the above-mentioned dissolution rate as measured by the dissolution test method described in “1. Oral solid preparation.”

### BRIEF DESCRIPTION OF THE DRAWINGS

FIG. 1 shows an X-ray diffraction pattern of aripiprazole anhydrous crystal of Lot No, 1.

FIG. 2 shows an X-ray diffraction pattern of aripiprazole anhydrous crystal of Lot No, 2.

### EXAMPLES

The present invention is explained in detail below with reference to the Examples and Comparative Examples; however, the present invention is not limited thereto or thereby.

### Synthesis Example

### Synthesis of Crude Aripiprazole

A 1.7-fold volume of purified water, 2.0-fold mol of potassium carbonate (anhydrous), and a 7.5-fold volume of ethanol were added to 7-hydroxy-3,4-dihydro-2(1H)-quinolinone (7-HDC) (standard amount), and the mixture was stirred at 40° C. for about 0.5 hour. 1.1-fold mol of 4-chlorobutyl 2-nitrobenzene sulfonate (CBNBS) was added thereto and reacted at about 40° C. for about 5 hours. After the solvent was distilled off, a 25-fold volume or more of purified water was added to the residue, followed by washing while stirring at about 50° C. The crystal was separated and washed with purified water.

A 14-fold volume of purified water, 1.07-fold mol of potassium carbonate (anhydrous), and 1.0 mol of 1-(2,3-dichlorophenyl)piperazine monohydrochloride(2,3-DCPP) were added to the obtained wet crystal, and the mixture was reacted at 80° C. or more for about 4 hours. After cooling, the crystal was separated and washed with purified water. A 34-fold volume of ethyl acetate was added to the obtained wet crystal, and heated until solvent reflux to distill ethyl acetate and remove water, After cooling, ethyl acetate in an amount equivalent to the amount removed by distillation and 5 wt % of activated carbon were added thereto, and refluxed for 0.5 hour or more. The reaction mixture was hot-filtered to distill off a 20-fold volume of ethyl acetate, and then cooled. The crystal was separated, and dried at about 55° C. to obtain crude aripiprazole.

### Recrystallization Process

A 16-fold volume of ethanol and a 2-fold volume of purified water were added to the obtained crude aripiprazole, and heated until solvent reflux to completely dissolve the crude aripiprazole. A 2-fold volume of purified water was added thereto and heated again until reflux, and then stirred for 30 minutes or more under reflux. Subsequently, the resultant was cooled to 10° C. or less to fully deposit a crystal, and then solid liquid separation was performed to obtain a crystal. The obtained crystal was dried at 80° C. for 24 hours.

### Milling Step

Aripiprazole obtained from recrystallization after drying was milled using a hammer mill for experiment (labo mill: Fuji Paudal Co., Ltd., model LM-05) under the following conditions.

### Milling Conditions

Main axis rotation rate: 12000 rpm
Screen: 5 mmHB
Introduction speed: About 20 g/min

The properties of anhydrous crystal (Lot No, 1) synthesized in the synthesis example were measured and evaluated according to the following method.

### X-Ray Diffraction Measurement

Using a X-ray diffractometer (08 ADVANCE) produced by Bruker AXS, X-ray diffraction patterns in the range of a diffraction angle of 3 to 40° were measured according to a powder X-ray diffraction method, Copper was used as an anticathode.

### Hygroscopic Test

About 1 g of aripiprazole obtained in the Synthesis Example was weighed in each of the weighing bottles having a diameter of about 5 cm. Each of the bottles was sealed with Kimwipe, and allowed to stand in a desiccator including water. The desiccator was placed in an oven at 60° C., and allowed to stand for 24 hours.

The weighing bottles were removed from the desiccator, and allowed to stand for 16 hours or more in a desiccator (room temperature/about 30% RH) including an MgCl₂/6H₂O saturated salt solution, thereby removing attached excess water. The water contents of the samples before and after the hygroscopic test were measured according to the following test procedure.

### Water Content Measurement Method

0.1 g of the aripiprazole obtained in the Synthesis Example and 0.1 g of the aripiprazole obtained after the hygroscopic test were accurately weighed. Using a moisture meter (CA-200) produced by Mitsubishi Chemical Analytech Co., Ltd., a test was performed according to the coulometric titration method, which is a water content measurement method (Karl Fischer method).

### Melting Point Measurement

The melting point was measured by the following procedure using a melting point measurement apparatus (B-545) produced by BUCHI. A sample was introduced into a capillary tube, one end of which was closed, to a height of 2.5 to 3.5 mm from the bottom of the capillary tube. A block was heated to 130° C., and the capillary tube was introduced into the block. The block was heated at 3° C./min between 130 to 135° C. and 1° C./min between 135° C. to melting point, and the temperature at which the sample was completely dissolved was measured.

The properties of the anhydrous crystal (Lot No. 2) synthesized according to the same method as the method of the Synthesis Example were measured in the same manner as above and evaluated.

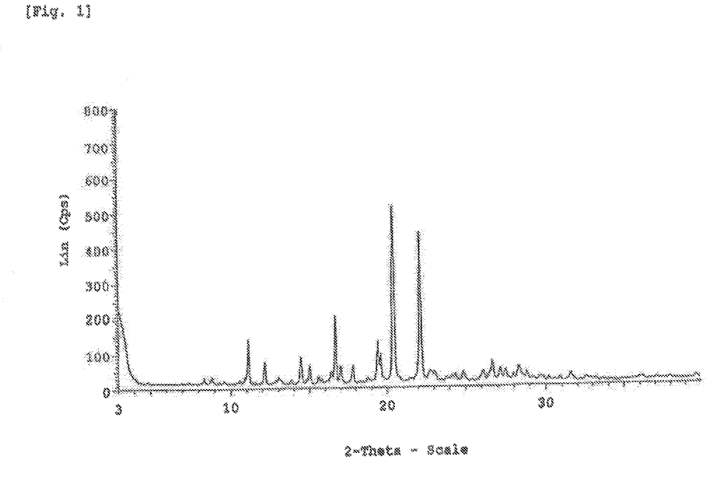

Table 1 shows the measurement results. FIG. 1 (Lot No. 1) and 2 (Lot No. 2) show the X-ray diffraction pattern obtained by the X-ray diffraction measurement,

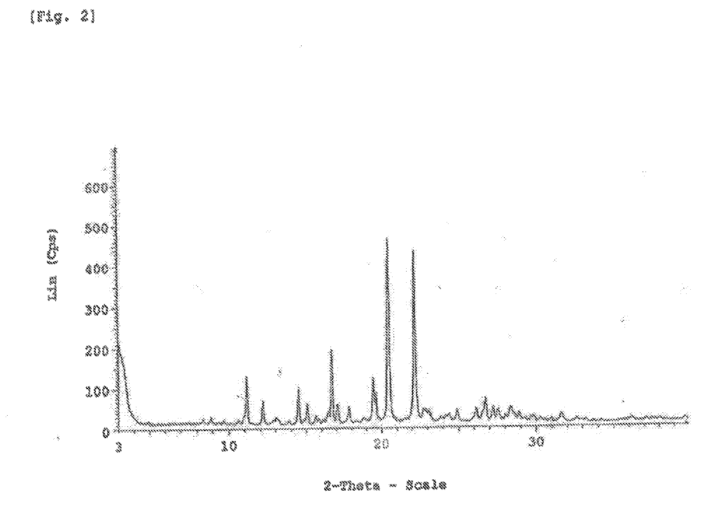

TABLE 1

| | Lot No. 1 | Lot No. 2 |
|---|---|---|
| Powder X-ray | Diffraction peak | Diffraction peak |
| diffraction | was recognized at | was recognized at |
| | 2θ = 11.09°, | 2θ = 11.13°, |
| | 16.64°, 20.43°, | 16.69°, 20.47°, |
| | 22.11° (FIG. 1) | 22.19° (FIG. 2) |
| Water content | 0.003 | 0.032 |
| level (wt %) before | | |
| humidification | | |
| Water content | 0.809 | 2.759 |
| level (wt %) after | | |
| humidification | | |
| Melting point (° C.) | 139.7 | 139.8 |

By the measurements under the test conditions shown above, the formation of aripiprazole anhydrous Type I (highly hygroscopic aripiprazole anhydrous crystal) was confirmed in the obtained crystals.

### Reference Examples 1 to 4

### Finely Milled Powder of Highly Hygroscopic Aripiprazole Anhydrous Crystal

Highly hygroscopic aripiprazole anhydrous crystals of Lot Nos, 1 and 2 obtained in the Synthesis Example above were milled using a hummer mill (main axis rotation rate: 1200 rpm, screen: 5 mm herringbone, introduction speed: about 20 g/min). Then, dry milling was performed using a jet mill (spiral jet mill (spiral JM) or AO jet mill (AOJM)), air pressures (forcing pressure and milling pressure), and a feed rate shown in Table 2.

The particle size of the resulting finely milled powder of highly hygroscopic aripiprazole anhydrous crystal was measured using Dry Unit 2000, which was a laser diffraction scattering particle size distribution analyzer (Laser Micron Sizer (LMS-2000e)), and the mean particle size was calculated from the obtained measurement results. Table 2 shows the measurement results. The measurement conditions using a laser diffraction scattering particle size distribution analyzer were as follows: Particle refractive index: 1.750, imaginary part: 0.1, and dispersive pressure: 0.15 MPa.

The finely milled powder obtained above was evaluated by a hygroscopic test according to the following method.

About 1 g of milled powder was weighed in a weighing bottle with a diameter of 5 cm, allowed to stand in water/desiccator in which the temperature and humidity were respectively set to 60° C. and 100%, and then allowed to stand in MgCl₂.6H₂O saturated salt solution/desiccator having a room temperature and humidity of about 30% for 24 hours, Thereafter, the water content was measured. The water content was measured according to the Karl Fischer method by accurately measuring 0.1 g of a sample.

Table 3 shows the measurement results. Each of the samples of finely milled powders measured showed 0.4% or more.

TABLE 2

| | | Reference No. | | | |
|---|---|---|---|---|---|
| | | Reference | Reference | Reference | Reference |
| | | Example 1 | Example 2 | Example 3 | Example 4 |
| | | Milling material powder | | | |
| | | Lot No. 1 | Lot No. 1 | Lot No. 2 | Lot No. 2 |
| Milling | Forcing | 0.6 | 0.5 | 0.4 | 0.4 |
| conditions | pressure | | | | |
| | (MPa) | | | | |
| | Milling | 0.45 | 0.55 | 0.3 | 0.3 |
| | pressure | | | | |
| | (MPa) | | | | |
| | Feed rate | 40 | 30 | 120 | 170 |
| | (g/h) | | | | |
| | Jet mill | Spiral JM | AO JM | AO JM | AO JM |
| | used | | | | |
| | Mean | 2.6 | 5.1 | 8.9 | 9.6 |
| | particle | | | | |
| | size (μm) | | | | |

### Examples 1 to 4

### Method for Producing Tablets and Method for Stability Test (Containing Dissolution Test)

Tablets were formed using the finely milled powders prepared in Reference Examples 1 to 4 above.

The jet-milled aripiprazole finely milled powder (highly hygroscopic aripiprazole anhydrous crystal) (Type 1) (9 g), directly compressible lactose (207 g), corn starch (30 g), crystalline cellulose (30 g), and hydroxypropyl cellulose (6 g) were introduced into a high speed granulator, and mixed for about 10 minutes by rotating blades at about 500 rpm. Thereafter, magnesium stearate was added in an amount of 1 wt % per tablet and mixed to prepare a powder for tableting. The powder for tableting was supplied to a tableting machine (Virgo 12HUK, produced by Kikusui Seisakusho Ltd.), and formed into tablets (tablets of Examples 1 to 4) each having a weight of 95 mg using a corner flat die punch having a diameter of 6.0 mm.

### Test Example 1 (Tablet Stability Test)

Each of the tablets of Examples 1 to 4 was allowed to stand under opening conditions at 40° C./75% RH for one week, two weeks, or one month. The dissolution rate was measured according to the following method.

### Dissolution Test Method

An acetic acid buffer (500 ml) with a pH of 5.0 was used as a test liquid, and one tablet was introduced into the test liquid, thereby obtaining a test solution. Using the resulting test solution, the test was performed at 75 rpm according to the second paddle method of the dissolution test method. The acetic acid buffer mentioned above was prepared as 1000 ml of the acetic acid buffer by dissolving acetic acid (1.97 g) and sodium acetate trihydrate (9.15 g) in water.

The dissolution liquids obtained 10, 20, 30, 45, and 60 minutes after the beginning of the test were respectively referred to as Test Solution T10, T20, T30, T45, and T60.

As a standard solution, an aripiprazole standard product (0.05 g) was accurately weighed, and ethanol (95%) was added and dissolved to obtain exactly 50 ml of a solution. 6 ml of the solution was accurately weighed, and a test liquid was added thereto to obtain exactly 1000 ml of solution.

Each of the test solutions and standard solution was passed through a filter having a pore size of 10 or 20 μm. Each of the filtrates was introduced into a spectrophotometer equipped with a flow cell (cell length of 10 mm), and a test was performed according to an UV visible absorption spectrometry. As and At10, At20, At30, At45, and At60, which were differences between the absorbance at a wavelength of 249 nm and the absorbance at a wavelength of 325 nm, were individually measured.

Each of the Test Solutions T10, T20, T30, and T45 after measurement was placed back into individual test containers. The same operation was performed for the other five samples.

As a dissolution device, any test device that applies to the paddle method harmonized trilaterally by the US, EU, and JP can be used; however, the device shown below was used. Model: NTR-6200A, produced by Toyama Sangyo Co., Ltd.

The dissolution rate (%) relative to the indicated amount of aripiprazole was determined by the following formula.

Dissolution Rate (%) relative to the indicated amount of aripiprazole=Amount of the aripiprazole standard product (mg)×At×As×2

Herein, As indicates a difference between the absorbances of the standard solution, and At indicates At10, At20, At30, At45, or At60.

Table 3 shows the dissolution rate of each sample obtained 60 minutes after the start of the dissolution test. One sample was not allowed to stand (initial value), and the other samples were allowed to stand under the opening conditions of 40° C./75% RH for one week, two weeks, and one month.

TABLE 3

| | | Example | | | |
|---|---|---|---|---|---|
| | | Example 1 | Example 2 | Example 3 | Example 4 |
| | | Finely milled aripiprazole crystal | | | |
| | | Reference | Reference | Reference | Reference |
| | | Example 1 | Example 2 | Example 3 | Example 4 |
| Mean particle size of | | 2.6 | 5.1 | 8.9 | 9.6 |
| aripiprazole (μm) | | | | | |
| Hygroscopic test of | | | | | |
| finely milled | | | | | |
| aripiprazole crystal | | | | | |
| Water content | 0 hour | 0.10 | 0.21 | 0.24 | 0.29 |
| level (wt %) | 24 hours | 3.37 | 3.69 | 3.97 | 3.90 |
| Dissolution | 60 min | 92.26 | 97.33 | 93.36 | 93.05 |
| rate (%) | | | | | |
| Initial value | | | | | |
| Dissolution | 60 min | 88.09 | 88.99 | 77.78 | 76.09 |
| rate (%) | | | | | |
| 40° C./75% RH | | | | | |
| Opening | | | | | |
| conditions | | | | | |
| Stored for | | | | | |
| one week | | | | | |
| Dissolution | 60 min | 88.88 | 88.49 | 75.37 | 75.96 |
| rate (%) | | | | | |
| 40° C./75% RH | | | | | |
| Opening | | | | | |
| conditions | | | | | |
| Stored for | | | | | |
| two weeks | | | | | |
| Dissolution | 60 min | 86.45 | 87.23 | 74.68 | 75.37 |
| rate (%) | | | | | |
| 40° C./75% RH | | | | | |
| Opening | | | | | |
| conditions | | | | | |
| Stored for | | | | | |
| one month | | | | | |

### Comparative Example 1

Using the aripiprazole anhydrous crystal (Lot No. 1) milled with a hammer mill, the mean particle size was calculated in the same manner as Example 1. The mean particle size of the obtained finely milled powder was 27.2 μm. The hygroscopic test of the obtained finely milled powder was performed in the same manner as Example 1. As a result of the hygroscopic test, the water content at 0 hour was 0.00 wt %; however, the water content after 24 hours was 0.49 wt %.

Using the crystal of the obtained finely milled powder, tablets were produced in the same manner as Example 1. The stability test of the obtained tablets was performed in the same manner as in the above Examples. The results indicated that the dissolution rate (%) was 78.67% as the initial value, 42.66% after one week of storage, 41.04% after two weeks of storage, and 41.75% after one month of storage.

### EXAMPLE

Tablets were prepared in the same manner as Example 1, except that the components of the tablets were changed to the following. A stability test (including a dissolution test) was performed. Table 4 shows the results of the stability test (dissolution test).

The jet-milled aripiprazole finely milled powder (highly hygroscopic aripiprazole anhydrous crystal) (Type I) (9 g) obtained in Reference Example 1, directly compressible lactose (114.0 g), crystalline cellulose (114.0 g), sodium carboxymethyl starch (21.0 g), carmellose calcium (21.0 g), and light anhydrous silicic acid (3.0 g) were mixed, and then magnesium stearate (3.0 g) was added thereto and mixed. The mixture was then supplied to a tableting machine, and formed into tablets using a die punch.

TABLE 4

| | | Example |
|---|---|---|
| | | Example 5 |
| | | Finely milled |
| | | aripiprazole crystal |
| | | Reference |
| | | Example 1 |
| Mean particle size of | | 2.6 |
| aripiprazole (μm) | | |
| Hygroscopic test of | | |
| finely milled | | |
| aripiprazole crystal | | |
| Water content | 0 hour | 0.10 |
| level (wt %) | 24 hours | 3.37 |
| Dissolution | 60 min | 77.46 |
| rate (%) | | |
| Initial value | | |
| Dissolution | 60 min | 75.40 |
| rate (%) | | |
| 40° C./75% RH | | |
| Opening | | |
| conditions | | |
| Stored for | | |
| one week | | |
| Dissolution | 60 min | 76.09 |
| rate (%) | | |
| 40° C./75% RH | | |
| Opening | | |
| conditions | | |
| Stored for | | |
| two weeks | | |
| Dissolution | 60 min | 75.74 |
| rate (%) | | |
| 40° C./75% RH | | |
| Opening | | |
| conditions | | |
| Stored for | | |
| one month | | |

1-6. (canceled)

7. A method for producing an oral solid preparation comprising the steps of:
(1) milling a highly hygroscopic aripiprazole anhydrous crystal that has a hygroscopicity such that the water content after being allowed to stand in a desiccater at a temperature of 60° C. and a humidity of 100% for 24 hours is 0.809% or more, into a finely milled powder having a mean particle size of 1 to 10 μm, wherein the mean particle size is the volume mean diameter as calculated from the particle size distribution measured by a laser scattering particle size distribution analyzer;
(2) mixing the obtained finely milled powder with a pharmaceutically acceptable carrier, and
(3) compressing the mixture obtained in step (2) into a tablet.

8. The method for producing an oral solid preparation according to claim 7, wherein the milling in step (1) is performed by dry milling method.

9. The method for producing an oral solid preparation according to claim 7, wherein the compressing in step (3) is performed by direct powder compression method.
